(19) United States
(12) Patent Application Publication
(10) Pub. No.: US 2023/0271023 A1
(43) Pub. Date: Aug. 31, 2023

(54) Magnetic Resonance System and Method for Weakening and Expelling Living Coronavirus from a Patient Body

(71) Applicant: Svyatoslav Orlov-Kurylov, Scarborough (CA)

(72) Inventor: Svyatoslav Orlov-Kurylov, Scarborough (CA)

(21) Appl. No.: 18/152,301

(22) Filed: Jan. 10, 2023

Related U.S. Application Data
(60) Provisional application No. 63/300,812, filed on Jan. 19, 2022.

(51) Int. Cl. A61N 2/00; A61L 9/14

(52) U.S. Cl. CPC A61N 2/004; A61L 9/145

(57) ABSTRACT

The present invention relates to a novel COVID-19 treatment system and associated method. The system features an electromagnetic field pulse generator for generating a low frequency electromagnetic field pulse configured to create a magnetic resonance with the magnetic field generated by the living coronavirus in and around a patient body of an infected person. The magnetic resonance weakens the coronavirus and expels the coronavirus from the patient body to an outside chamber wherein the chamber air is sanitized by passing through a disinfectant solution. In some embodiments, the electromagnetic field generated by the generator may include frequencies from radio frequencies to higher millimeter waves to microwaves.

### CROSS REFERENCE TO RELATED APPLICATION

The present application claims priority to, and the benefit of, U.S. Provisional Application No. 63/300,812, which was filed on Jan. 19, 2022, and is incorporated herein by reference in its entirety.

### FIELD OF THE INVENTION

The present invention generally to the field of virus sterilization. More specifically, the present invention relates to a novel system and method for expelling living coronavirus from a patient using electromagnetic pulse field. The system generates an electromagnetic field having a frequency that coincides with the coronavirus frequency created around the body such that a magnetic resonance is created for weakening and expelling the coronavirus from the patient’s body. The method is easy, safe, and effective on all mutants and variants of coronavirus. Accordingly, the present disclosure makes specific reference thereto. Nonetheless, it is to be appreciated that aspects of the present invention are also equally applicable to other like applications, devices, and methods of manufacture.

### BACKGROUND

By way of background, Coronavirus disease (COVID-19) is an infectious disease caused by the SARS-CoV-2 virus. The virus usually spreads from an infected person’s mouth or nose in small liquid particles when the infected person coughs, sneezes, speaks, sings, or breathes. These particles range in size from larger respiratory droplets to smaller aerosols. Generally, individuals protect themselves and others from infection by staying at least 1 meter apart from others, wearing a properly fitted mask, washing hands, and/or using an alcohol-based sanitizer gel/rub/lotion on the hands frequently.

Many individuals may develop severe symptoms such as joint or muscle pain, heart symptoms, blood clots, and more. Individuals who have severe illness with COVID-19 may experience organ damage affecting the heart, kidneys, skin, and brain. Inflammation and problems with the immune system may also happen. Immunization of individuals is a common method deployed at large scale by authorities to prevent coronavirus infection. Use of vaccines is used globally for immunization and preventing transmission of COVID-19. All the vaccines contain an active antigen component which generates an immune response, or the blueprint for making the active component. The antigen may be a small part of the disease-causing organism, like a protein or sugar, or it may be the whole organism in a weakened or inactive form. The vaccines used for preventing COVID-19 and other COVID prophylactics and treatments are based on administration of different chemical agents (such as antigen and other components such as stabilizers) in a human body. However, such chemical agents can have adverse and side effects on humans. Side effects, both minor and major, are possible due to use of such chemical agents. Side effects ranging from fever, or pain and redness at the injection site are common.

Also, most of the modern methods are used for preventing a human body from contracting coronavirus. Removing coronavirus from a human body requires expensive, invasive, and complex medical procedures. Both individuals and authorities desire an improved COVID-19 treatment method for curing and expelling coronavirus from a human body.

Therefore, there exists a long-felt need in the art for a COVID-19 treatment method that does not administer chemical agents in a human body. There is also a long-felt need in the art for a COVID-19 treatment method that obviates introduction of vaccines into human bodies. Additionally, there is a long-felt need in the art for a COVID-19 treatment method that is non-invasive and safe to use. Moreover, there is a long-felt need in the art for a COVID-19 treatment method that does not cause any minor or major side effects. Further, there is a long-felt need in the art for a COVID-19 treatment method that both prevents and treats coronavirus infection. Furthermore, there is a long-felt need in the art for a COVID-19 treatment method that uses resonance signaling for treating coronavirus in an infected person. Finally, there is a long-felt need in the art for a COVID-19 treatment method that weakens the coronavirus and expels same from treatment administered outside or exterior of the patient.

The subject matter disclosed and claimed herein, in one embodiment thereof, comprises a system and associated method for expelling coronavirus from body of a coronavirus infected patient. The system features an enclosed chamber, an electromagnetic field generator for generating an electromagnetic field, an electromagnetic field distributor for uniformly distributing the generated electromagnetic field above a patient lying on a bed positioned under the electromagnetic field distributor, an air-conditioner for blowing fresh air into the chamber, a fluid disinfectant chamber for purifying air of the chamber; wherein the electromagnetic field is configured within the virus’ resonant frequency to expel the coronavirus from the body of the patient into the chamber. The frequency of the low frequency electromagnetic field coincides with the electromagnetic field generated by the coronavirus for creating magnetic resonance and weakening the coronavirus.

In this manner, the COVID-19 resonance treatment method of the present invention accomplishes all of the forgoing objectives and provides users with a treatment method for expelling living coronavirus from a patient using an electromagnetic pulse field. Electromagnetic field frequency applied around a patient matches with the frequency produced by coronavirus and thus, attracts the virus outside and expels same out from the body. The method does not use chemical agents, vaccines and more for an effective, easy, and non-invasive treatment of coronavirus.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a system for expelling coronavirus from the body of a coronavirus infected patient. The system further comprising an electromagnetic field generator configured for generating a low frequency Pulsed Electro-Magnetic Field, an electromagnetic field distributor for uniformly distributing the generated low frequency electromagnetic field around a patient lying on a bed positioned under the electromagnetic field distributor, wherein the bed, the generator, and the distributor are positioned inside a chamber; an air-conditioner for blowing fresh air into the chamber; a fluid disinfectant chamber for purifying air of the chamber; wherein the low frequency electromagnetic field is configured for weakening the coronavirus inside the patient and expelling the coronavirus into the chamber. The frequency of the low frequency electromagnetic field coincides with the electromagnetic field generated by the coronavirus.

The subject matter disclosed and claimed herein, in one embodiment thereof, comprises a method for weakening coronavirus in a patient’s body and expelling coronavirus from the patient’s body. The method comprising the steps of generating a low frequency electromagnetic field inside a chamber using an electromagnetic field generator, controlling frequency of the electromagnetic field using a controller, distributing the generated electromagnetic field around a patient lying on a bed inside the chamber, penetrating the generated electromagnetic field in the patient body, weakening the electromagnetic field generated inside the body of the patient by coronavirus infecting the patient, expelling the coronavirus from the patient body into chamber air, and passing the chamber air through a disinfectant solution for sanitizing the chamber air.

In another aspect, the present invention provides a COVID-19 magnetic resonance treatment method of treating viruses, specifically coronavirus by generating a magnetic resonance inside the body of a coronavirus patient. The method includes low frequency electromagnetic field around the patient and penetrate inside the patient, interaction of the low frequency electromagnetic field with the magnetic field produced by coronavirus, generation of magnetic resonance between said fields when the frequency of the low frequency electromagnetic field and the magnetic field of the coronavirus coincides, and weakening and expelling coronavirus from the patient body to outside air wherein the air is sanitized using a disinfectant chamber.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a COVID-19 resonance treatment system of the present invention in accordance with the disclosed architecture; and

FIG. 2 illustrates a flow diagram describing a process of treating coronavirus using the COVID-19 resonance treatment system of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a COVID-19 treatment method that does not administer or inject chemical agents into a human body. There is also a long-felt need in the art for a COVID-19 treatment method that obviates use of vaccines on human bodies. Additionally, there is a long-felt need in the art for a COVID-19 treatment method that is non-invasive and safe to use. Moreover, there is a long-felt need in the art for a COVID-19 treatment method that does not cause any minor or major side effects. Further, there is a long-felt need in the art for a COVID-19 treatment method that both prevents and treats coronavirus. Furthermore, there is a long-felt need in the art for a COVID-19 treatment method that uses resonance signaling for treating coronavirus in an infected person. Finally, there is a long-felt need in the art for a COVID-19 treatment method that weakens the coronavirus and expels same from treatment administered outside or exterior of the patient.

The present invention, in one exemplary embodiment, is a method for weakening coronavirus in a patient’s body and expelling coronavirus from the patient’s body. The method comprising the steps of generating a low frequency electromagnetic field inside an enclosed chamber using an electromagnetic field generator, controlling frequency of the electromagnetic field using a controller, distributing the generated electromagnetic field around a patient lying on a bed inside the chamber, penetrating the generated electromagnetic field in the patient body, weakening the electromagnetic field generated inside the body of the patient by coronavirus infecting the patient by creating a magnetic resonance, expelling the coronavirus from the patient body into chamber air, and passing the chamber air through a disinfectant solution for sanitizing the chamber air.

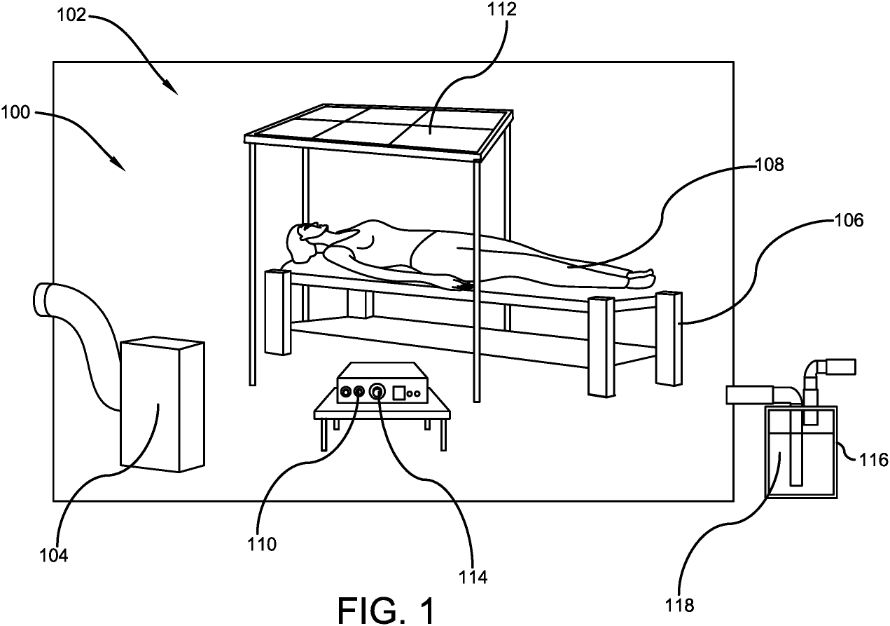

Referring initially to the drawings, FIG. 1 illustrates a perspective view of one potential embodiment of a COVID-19 resonance treatment system of the present invention in accordance with the disclosed architecture. The COVID-19 resonance treatment system 100 of the present invention is designed for treating viruses, specifically coronavirus by using an electromagnetic field around a patient within an air-conditioned chamber. More specifically, the system 100 includes an air-conditioned chamber 102 which can be in the form of an enclosed space such as a room or a chamber. The chamber 102 includes an air-conditioner 104 for providing a surrounding temperature from about 60° F. to about 80° F. The air-conditioner 104 can be any conventional air-conditioner for providing a uniform temperature and cool air.

For expelling coronavirus from a human body, a flat bench or bed 106 is positioned in the chamber 102 for a patient to lie down in supine or prone position. The air-conditioner 104 is oriented to blow fresh or cool air towards the bed 106 for maintaining a cool temperature on the bed 106. The system 100 is specifically designed for using electromagnetic field resonance therapy for expelling coronavirus from a human body wherein a human 108 lies down on the bed 106. An electromagnetic field generator 110 is coupled to an electromagnetic field distributor 112 for creating a uniform electromagnetic field around the body of the human 108. In the preferred embodiment, the generator 110 is configured to produce Pulsed Electro-Magnetic Field (PEMF) for stimulating cells of the human body and weakening the coronavirus present inside the body of the patient 108.

A frequency modulator 114 disposed on the generator 110 is used for adjusting frequency of the generated electromagnetic field such that the frequency of the produced electromagnetic field coincides with the frequency of the coronavirus. The electromagnetic field generated by the generator 110 has low frequency in the range from about 4 Hz to about 10 Hz and is configured to weaken the coronavirus while also reducing hyper-inflammation and mortality caused by COVID-19 infection. The Pulsed Electromagnetic Field (PEMF), which has the same frequency as coronavirus particles, attracts the coronavirus from a human body to outside, therefore, effective in weakening and expelling coronavirus from the body. The electromagnetic field generated by the generator may have a magnetic field intensity in the range from about 0.5 T (Tesla) to about 2 T, which is corresponding to the distance between the patient’s body 108 and the electromagnetic field distributor 112, including from about 1 foot to about 4 feet therebetween.

The chamber 102 is sealed and enclosed with no contact from outside and includes a fluid disinfectant chamber or container 116 storing disinfectant solution 118 for disinfecting the chamber 102. The fluid disinfectant chamber or container 116 is configured to receive air from chamber 102 and disinfectant to eliminate any traces of coronavirus present in the chamber 102. The coronavirus is expelled from the human body 108 to the chamber 102. Air from the chamber 102, in real time or after the therapy process, may be sanitized using the fluid disinfectant chamber or container 116.

It should be noted that the COVID-19 treatment system 100 of the present invention can be deployed in any medical facility for treating patients contaminated with coronavirus in a non-invasive manner. Further, no chemical agent is administered in a human body and thereby obviates side-effects therefrom. The system 100 can be deployed without any technical expertise and can be easily monitored to coincide the electromagnetic field generated by the generator 110 and the field generated inside the human body by the coronavirus.

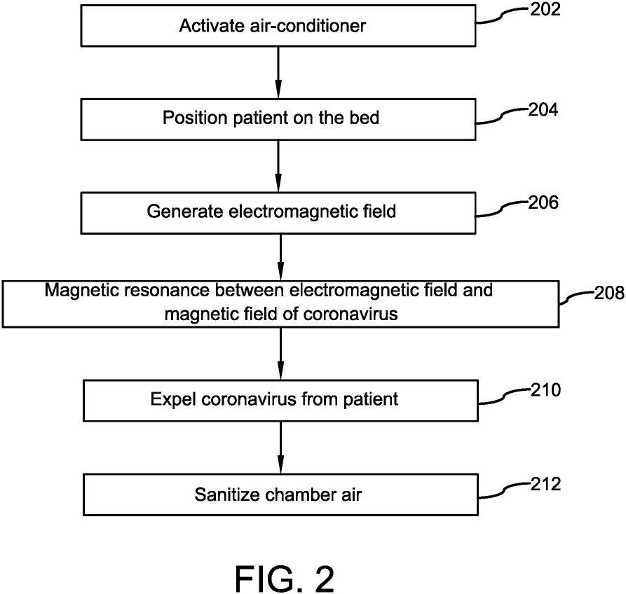

FIG. 2 illustrates a flow diagram describing a process of treating coronavirus using the COVID-19 resonance treatment system of the present invention in accordance with the disclosed architecture. Initially, an air-conditioner is activated for maintaining a cool or comfortable temperature in the chamber (Step 202). Then, a patient infected with coronavirus lies down on the bed, preferably in a supine position (Step 204). Preferably, the patient wears a hospital gown allowing effective penetration of electromagnetic field inside the patient body. Thereafter, electromagnetic field is generated by activating the generator such that electromagnetic field is distributed uniformly around the patient and penetrates the body of the patient (Step 206).

Then, a generated electromagnetic field interacts with the magnetic field generated by the coronavirus such that the coronavirus is weakened (Step 208). Finally, the coronavirus is expelled from the body of the patient to the chamber (Step 210). For cleaning the chamber, the air from the chamber is passed through a disinfecting solution for sterilization of the chamber air (Step 212). The low frequency electromagnetic field is also configured to increase peripheral oxygen saturation levels in coronavirus patients.

In some embodiments of the present invention, the low frequency magnetic field is safe for humans and is non-ionizing. When the frequency of the electromagnetic field coincides with the electromagnetic/magnetic field of the coronavirus, a magnetic resonance may be created to rupture the coronavirus. In some embodiments of the present invention, the electromagnetic field generated by the generator may include frequencies from radio frequencies to higher millimeter waves to microwaves.

Certain terms are used throughout the following description and claims to refer to particular features or components. As one skilled in the art will appreciate, different persons may refer to the same feature or component by different names. This document does not intend to distinguish between components or features that differ in name, but not structure or function. As used herein “system”, “COVID-19 resonance treatment system”, “COVID-19 treatment system”, “coronavirus treatment system”, and “coronavirus weakening system” are interchangeable and refer to the COVID-19 magnetic resonance treatment system 100 of the present invention.

Notwithstanding the forgoing, the COVID-19 magnetic resonance treatment system 100 of the present invention can be of any suitable size and configuration as is known in the art without affecting the overall concept of the invention, provided that it accomplishes the above-stated objectives. One of ordinary skill in the art will appreciate that the COVID-19 magnetic resonance treatment system 100 as shown in the FIGS. are for illustrative purposes only, and that many other sizes and shapes of the COVID-19 magnetic resonance treatment system 100 are well within the scope of the present disclosure. Although the dimensions of the COVID-19 magnetic resonance treatment system 100 are important design parameters for user convenience, the COVID-19 magnetic resonance treatment system 100 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A coronavirus resonance treatment system comprising:
a chamber;
an air conditioner;
an electromagnetic (EM) pulse generator;
an EM distributor; and
a frequency controller;
wherein said chamber is comprised of a platform that is sized and configured to receive a prone placement of a patient;
wherein said air conditioner supplies a flow of air to said chamber;
wherein said EM pulse generator generates EM pulses; and
further wherein said EM distributor distributes said EM pulses to said chamber and around the patient for treating a coronavirus.

2. The coronavirus resonance treatment system of claim 1, wherein said chamber is a sealed enclosed space.

3. The coronavirus resonance treatment system of claim 2, wherein the flow of said air is at a temperature ranging from about 60° F. to about 80° F.

4. The coronavirus resonance treatment system of claim 3, wherein said coronavirus is COVID-19.

5. The coronavirus resonance treatment system of claim 4, wherein said platform is comprised of a flat bench.

6. The coronavirus resonance treatment system of claim 5, wherein said EM pulses are Pulsed Electro-Magnetic Field (PEMF) pulses for expelling the coronavirus particles from a human body and for stimulating cells of the patient.

7. The coronavirus resonance treatment system of claim 6, wherein said frequency controller is integrated with said EM pulse generator for adjusting a frequency of said plurality of EM pulses to coincide with a frequency of the coronavirus inside the human body of the patient.

8. The coronavirus resonance treatment system of claim 7, wherein a frequency of said plurality of EM pulses is in a range from about 4 Hz to about 10 Hz.

9. The coronavirus resonance treatment system of claim 8, wherein said PEMF pulses have a magnetic field intensity in the range from about 0.5 T to about 2 T, corresponding to a distance between the patient’s body and said EM distributor in the range from about 1 foot to about 4 feet.

10. The coronavirus resonance treatment system of claim 9 further comprises a fluid disinfectant container for storing a disinfectant solution to disinfect said chamber.

11. The coronavirus resonance treatment system of claim 10, wherein said fluid disinfectant container receives air from said chamber, disinfects said air, and expels said air to the surrounding area.

12. A method for treating a patient infected with a coronavirus, the method comprising the steps of:
providing a coronavirus resonance treatment system having a chamber, a platform, an air conditioner, an electromagnetic (EM) pulse generator, an electromagnetic (EM) distributor, and a frequency controller;
placing a patient in a prone position onto said platform in said chamber, wherein said chamber is a sealed enclosed space;
activating said air conditioner to supply a flow of air to said chamber;
generating EM pulses with said EM pulse generator; and
distributing said EM pulses with said EM distributor to said chamber and around the patient for treating the coronavirus.

13. The method of treating a patient with coronavirus of claim 12, wherein the flow of said air has a temperature ranging from about 60° F. to about 80° F.

14. The method of treating a patient with coronavirus of claim 12, wherein said coronavirus is COVID-19.

15. The method of treating a patient with coronavirus of claim 12, wherein said EM pulses are Pulsed Electro-Magnetic Field (PEMF) pulses for expelling the coronavirus particles from a human body and for stimulating cells of a patient.

16. The method of treating a patient with coronavirus of claim 12, wherein said frequency controller integrated with said EM pulse generator for adjusting a frequency of said EM pulses to coincide with a frequency of the coronavirus inside the human body of the patient.

17. A method for treating a patient infected with coronavirus, the method comprising the steps of:
providing a coronavirus resonance treatment system having a chamber, an air conditioner, an electromagnetic (EM) pulse generator, an electromagnetic (EM) distributor, and a frequency controller;
placing a patient in said chamber in a prone position onto a platform, wherein said chamber is a sealed enclosed space;
activating said air conditioner to supply a flow of air to said chamber, wherein said flow of air has a temperature ranging from about 60° F. to about 80° F.;
generating EM pulses with said EM pulse generator, wherein a frequency of said plurality of EM pulses is in a range from about 4 Hz to about 10 Hz; and
distributing said EM pulses with said EM distributor to said chamber and around the patient for treating the coronavirus.

18. The method of treating a patient with coronavirus of claim 17, wherein said EM pulses are Pulsed Electro-Magnetic Field (PEMF) pulses for expelling coronavirus from a human body and for stimulating cells of the patient, and further wherein said PEMF pulses have a magnetic field intensity in the range from about 0.5 T to about 2 T.

19. The method of treating a patient with coronavirus of claim 18, wherein said coronavirus resonance treatment system further comprises a fluid disinfectant container for storing a disinfectant solution to disinfect said chamber.

20. The method of treating a patient with coronavirus of claim 19 further comprising the steps of:
receiving said air from said chamber into said fluid disinfectant container;
disinfecting said air; and
expelling said air to the surrounding area.
